(19) United States
(12) Patent Application Publication
(10) Pub. No.: US 2008/0221416 A1
(43) Pub. Date: Sep. 11, 2008

(54) System and method for detection of macular degeneration using spectrophotometry

(75) Inventor: Clark R. Baker, Newman, CA (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC

(21) Appl. No.: 11/716,776

(22) Filed: Mar. 9, 2007

(51) Int. Cl. A61B 3/10

(52) U.S. Cl. 600/318; 600/310

(57) ABSTRACT

Embodiments of the present invention relate to a system and method of detecting or monitoring macular degeneration in a patient. One embodiment of the present invention includes emitting a first light into the patient's retinal tissue at a first wavelength, emitting a second light into the patient's retinal tissue at a second wavelength, detecting the first and second lights after dispersion by the retinal tissue, and determining an amount of lipid proximate the retinal tissue based on the detected first and second lights.

### BACKGROUND OF THE INVENTION

1. Field of the Invention

The present invention relates generally to a method and system for detecting macular degeneration. Specifically, embodiments of the present invention relate to detecting and measuring changes in lipid content in and around retinal tissue to facilitate diagnoses and monitoring of macular degeneration.

2. Description of the Related Art

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

Macular degeneration is a leading cause of vision loss and encompasses several types of abnormalities in the macula of the eye. The macula is the portion of the retina that is located directly behind the lens. Cones, light-sensitive cells that are responsible for central vision, are heavily concentrated in the macula. In a healthy macula, the clear layer of the retina on the inside of the eye is nourished and maintained by an adjoining layer called the pigment epithelium. Behind the pigment epithelium is the choroid which contains the blood vessels that transport nourishment to and carry waste material away from the retina.

There are three major forms of macular degeneration: dry (also known as atrophic), wet (also known as disciform, exudative, or neovascular), and pigment epithelial detachment. The dry form, which occurs in more than 85% of AMD patients, leads to gradual vision loss and can be a precursor to the wet form. The dry form results from an inability of the pigment epithelium to digest the cone tips that the retina produces as waste materials. The pigment epithelium may swell and die as a result of the collection of undigested waste materials.

An early warning sign of dry macular degeneration is the formation of white or yellow spots, termed drusen, on the retina. Drusen are thought to be the fatty waste products from cone cells. Although used as an indicator of the development of macular degeneration, drusen are currently not treated. Instead, patients with drusen are closely monitored through regular eye exams. For example, patients may monitor their vision using the Amsler Grid, which consists of evenly spaced horizontal and vertical lines printed on black or white paper and a small dot is located in the center of the grid for fixation. While staring at the dot, a patient looks for wavy lines and missing areas of the grid. However, this test relies upon patient self-reporting of vision abnormalities and may thus be somewhat subjective. Macular degeneration may also be assessed by fluorescein dye-based imaging of the eye, which involves administering the dye into a patient's bloodstream. Such imaging techniques are associated with certain disadvantages, such as the time, effort, and expense involved in systemic administration of an imaging dye to a patient.

There exists a need for a fast, noninvasive technique for diagnosing and/or monitoring of the early signs of macular degeneration, since certain treatment options may have increased benefits for patients with early forms of macular degeneration.

### SUMMARY

Certain aspects commensurate in scope with the originally claimed invention are set forth below. It should be understood that these aspects are presented merely to provide the reader with a brief summary of certain forms of the invention might take and that these aspects are not intended to limit the scope of the invention. Indeed, the invention may encompass a variety of aspects that may not be set forth below.

There is provided a sensor that includes: a sensor body adapted for use associated with a patient's tissue; an emitter disposed on the sensor body, wherein the emitter is adapted to emit at least one wavelength of light between 850 nm and 1350 nm; and a detector disposed on the sensor body, wherein the detector is adapted to detect the wavelength of light.

There is provided a system that includes: a monitor; and a sensor adapted to be coupled to the monitor, the sensor including: a sensor body adapted for use associated with a patient's tissue; an emitter disposed on the sensor body, wherein the emitter is adapted to emit at least one wavelength of light between 850 nm and 1350 nm; and a detector disposed on the sensor body, wherein the detector is adapted to detect the wavelength of light.

There is provided a method of measuring lipid, or drusen, content in the retina that includes: emitting a light between 850 nm and 1350 nm into a tissue with an emitter; detecting the light; sending a signal related to the detected light to a processor; and determining a concentration of lipid or drusen in the retinal tissue

There is provided a method of manufacturing a sensor that includes: providing a sensor body adapted for use associated with a patient's tissue; providing an emitter disposed on the sensor body, wherein the emitter is adapted to emit at least one wavelength of light between 850 nm and 1350 nm; and providing a detector disposed on the sensor body, wherein the detector is adapted to detect the wavelength of light.

### BRIEF DESCRIPTION OF THE DRAWINGS

Advantages of the invention may become apparent upon reading the following detailed description and upon reference to the drawings in which:

FIG. 1 is a perspective view of a retinal lipid monitoring system in accordance with an exemplary embodiment of the present invention;

FIG. 2 is a side view of a sensor optically coupled to a patient's eye in accordance with an exemplary embodiment of the present invention;

FIG. 3 is a diagrammatic view of a patient's eye;

FIG. 4 is a schematic view of the sensor of FIG. 2 operating while optically coupled to a patient's eye;

FIG. 5 is a block diagram of a sensor in accordance with an exemplary embodiment of the present invention;

FIG. 6 is an attachment-side view of a non-invasive sensor in accordance with an exemplary embodiment of the present invention;

FIG. 7 is a cross-sectional, side view of an invasive sensor in accordance with an exemplary embodiment of the present invention;

FIG. 8 is a block diagram of a method in accordance with an exemplary embodiment of the present invention.

FIG. 9 is a block diagram of a system employing a spectrometer in accordance with an exemplary embodiment of the present invention; and

FIG. 10 is a block diagram of a method in accordance with an exemplary embodiment of the present invention.

### DETAILED DESCRIPTION OF SPECIFIC EMBODIMENTS

One or more specific embodiments of the present invention will be described below. In an effort to provide a concise description of these embodiments, not all features of an actual implementation may be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Embodiments of the present techniques relate generally to detecting macular degeneration using spectrophotometry to determine the presence of drusen in the eye. Specifically, the present techniques may include procedures and devices that facilitate diagnosis and/or monitoring of macular degeneration. A sensor according to the present techniques placed proximate to and/or within the eye may optically sense and measure the presence and/or concentration of drusen within the ocular tissue. For example, one embodiment may be utilized to detect drusen developing near the retina or macula with diffusely reflected near infrared spectroscopy (NIRS) that facilitates a determination of the presence of early macular degeneration. Further, the present techniques may include both invasive and non-invasive applications.

Sensors as provided herein may spectroscopically distinguish drusen from other structures in the eye, including water, at certain wavelengths in the infrared spectrum. Drusen are deposits of extracellular material that accumulate proximate to the retina, including the macula. Macular degeneration is generally associated with a build-up of additional drusen that may occur in two forms. Hard drusen are small, solid deposits, while soft drusen are larger and may have indistinct borders. Both hard and soft drusen contain a variety of cellular debris, lipids (fats), and minerals. The fats and proteins in drusen may be spectroscopically distinguished from the largely aqueous surrounding environment so that the presence of the drusen may be detected and quantified.

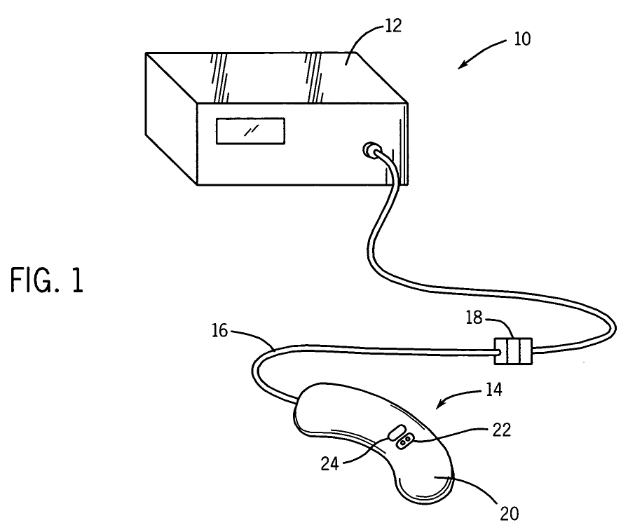

FIG. 1 is a perspective view of an ocular measurement system 10 that detects and quantifies the presence of retinal lipids in accordance with an exemplary embodiment of the present invention. The system 10 includes a monitor 12 (e.g., any suitable computer or signal processor) that communicatively couples to a sensor 14. The sensor 14 includes a sensor cable 16, a connector plug 18, and a body 20 configured to be used with a patient. The sensor 14 may couple directly to a patient's ocular tissue, or the sensor 14 may be placed proximate to the patient's ocular tissue. For example, in one embodiment, the sensor 14 may be non-invasive and the body 20 of the sensor 14 may be configured to externally couple to a patient's eye or to be placed proximate to, either touching or not touching, the tissue of the eye. In another embodiment, the sensor 14 may be invasive and have a body 20 that is configured to facilitate physical contact with the patient's eye tissue. The sensor cable 16 and connector plug 18 may enable electronic communication from the sensor 14 to the monitor 12, and facilitate coupling and/or decoupling of the sensor 14 from the monitor 12. In some embodiments, the sensor 14 couples directly to the monitor 12 via the sensor cable 16. Further, it should be noted that in some embodiments, the sensor 14 communicates with the monitor 12 wirelessly (e.g., via radio waves) and does not include the cable 16 or the connector plug 18.

The ocular measurement system 10 may be utilized to observe the drusen or other fatty deposits on the tissue of the eye to facilitate detection and/or monitoring of macular degeneration. This may be achieved spectroscopically by the system 10, because the absorbance of certain light wavelengths by these fatty deposits may correlate to their levels in the tissue of the eye. For example, a level of drusen may be estimated by emitting signals or waves into the patient's tissue and detecting the waves after dispersion and/or reflection by the tissue. For example, one embodiment of system 10 may emit light from a light source 22 (e.g., two or more light emitting diodes) into the eye and then detect the transmitted light with a light detector 24 (e.g., a photodiode or photo-detector) after the light has passed through the retinal tissue. The amount of transmitted light that passes through the retinal tissue may vary in accordance with varying amounts of constituents (e.g., fats) present in the tissue and the corresponding variance of light absorption characteristics. Accordingly, the amount of detected light may be correlated to an amount of drusen, which may be used to monitor or detect macular degeneration.

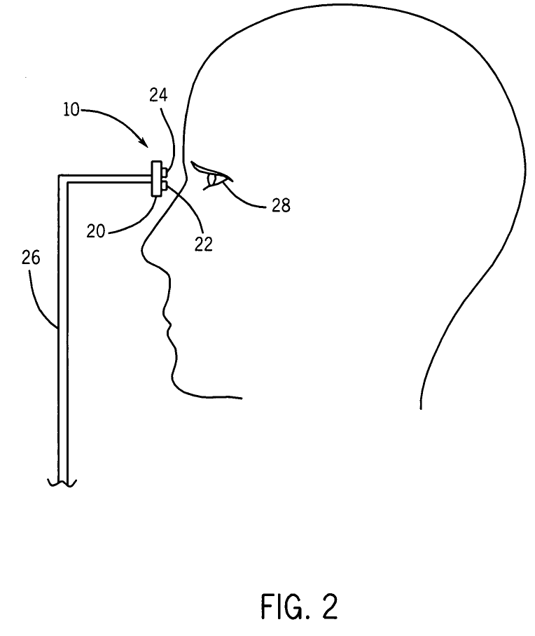

An exemplary sensor 14 appropriate for use for assessing the presence of drusen in the ocular tissue is shown in FIG. 2. FIG. 2 illustrates an exemplary reflectance-type sensor appropriate for use proximate to a patient's eye. The sensor 14 may be held in place by a substantially rigid positioning stand 26, which may be automatically or manually placed into position proximate to a patient's eye 28. The stand 20 may be suitably sized and shaped to position the sensor body 20 such that the emitter 22 and detector 24 are suitably close to the ocular tissue in order for the emitted light to shine through the lens and into the structures of interest in the eye.

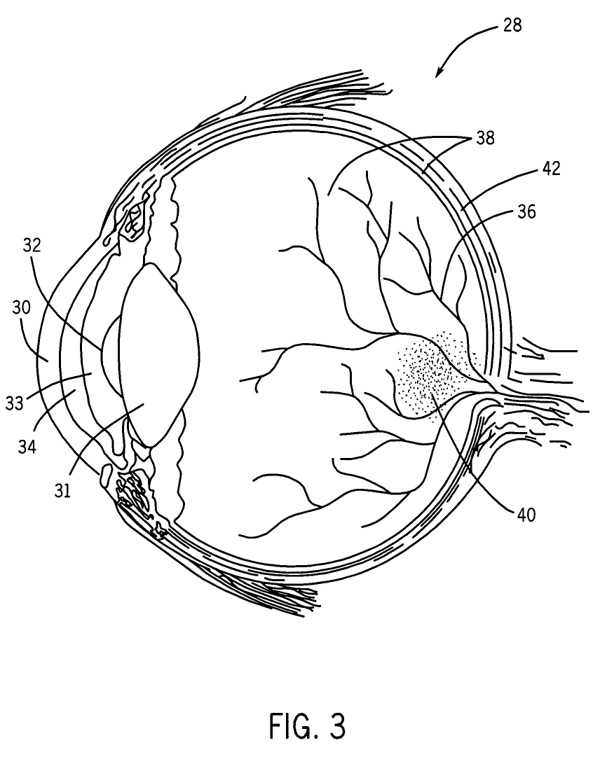

FIG. 3 is a two-dimensional cross-sectional representation of an exemplary eye 28. For the purposes of illustrating the principles of the present invention, it is beneficial to describe the structure and function of a few parts of the eye 28, namely, the cornea 30, the crystalline lens 31, the pupil 32 and the iris 33, the aqueous humor 34, the vitreous humor 35, the retinal blood vessels 36 and the retina 38, the macula 40, and the choroid 42.

The cornea 30 is the clear, transparent “window” of the eye. The cornea 30 is approximately 12 millimeters in diameter and typically varies from a little more than one half millimeter in thickness centrally to a little less than a millimeter at the edges. The cornea 30 consists of five distinct layers (from front to back): epithelium, Bowman's membrane, stroma, Descemet's membrane, and endothelium. The cornea 30 contains numerous tiny nerve fibers, but no blood vessels. The crystalline lens 31, along in cooperation with the cornea 30, provides for the focusing of light rays entering the eye 28. The iris 33 is the “colored part of the eye” (e.g., blue, brown, green, hazel, etc.). The iris 33 contains two major sets of muscles (for dilating and constricting the pupil) and numerous blood vessels and pigment cells and granules. The pupil 32 is the black “hole” or “space” in the center of the iris 120. The pupil 125 is not actually a structure or component of the eye 100, but an empty space, like an “open window.” The aqueous humor 34 is the thin, watery fluid that fills the space between the cornea and the iris. The vitreous humor 35 is the thin, watery fluid that fills the space between the iris and the retina. The cornea 30, pupil 32, iris 33, crystalline lens 31, aqueous humor 34, and vitreous humor 35 structures have very high water contents.

The retina 38 is the nerve cell layer of the eye 28 that functions much like the film in a camera. In short, the remainder of the eye 28 serves to focus light on to the retina 38 where photochemical reactions occur as part of the process of vision. The retina 38 is a thin, transparent tissue containing some 120 million separate rod cells (night vision) and 7 million cone cells (day and color vision) as well as millions of other structural supporting and interconnecting cells (collectively, the photoreceptor cells). The macula 40 is the sensitive, central, part of the retina that provides for sharp, detailed vision and contains the highest concentration of color-sensitive cone cells. The fovea (not shown) is the center of the macula 40. The retinal blood vessels 36 course through the retinal substance and, along with the underlying choroids 42, supply the necessary nutrients and oxygen for normal retinal function.

Embodiments of the present invention utilize reflectance NIRS to measure the presence of lipid-containing structures such as drusen in the ocular tissue. An increase or decrease in the drusen content of the ocular tissue generally produces unique alterations of the corresponding NIR (near infrared) reflectance spectrum in the wavelength range of 850-1350 nm. More specifically, fats, such as the drusen, absorb in the near infrared range, with a peak at 930 nm and a peak at 1210 nm. Accordingly, to detect and quantify drusen in the eye, the light source 22 of the sensor 14 may include one or more light emitting elements having wavelengths in the NIR range or ranges that are absorbed by the drusen. In specific embodiments, the wavelength or wavelengths may be in the range of 915-940 nm and/or 1160-1230 nm. For example, the sensor 14 may emit a first wavelength of about 930 nm and a second wavelength of about 1210 nm. In addition to emitting one or more wavelengths absorbed by drusen, the light source 22 may also emit one or more reference wavelengths that may be used by the monitor 12 to facilitate calculations relating to the detection and/or quantification of drusen in the eye.

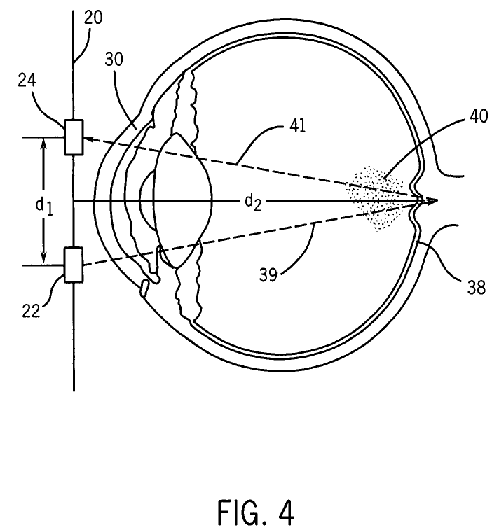

FIG. 4 illustrates a two-dimensional cross-sectional representation of an exemplary sensor 14 in operation. Light from the emitter 22, indicated by arrow 39, passes through the cornea 30, the crystalline lens 31, the pupil 32, the iris 33, and the aqueous humor 34. The wavelength or wavelengths of emitted light may be selected in order to minimize absorption by the water in these ocular structures, so as to assure that an adequate amount of light reaches and is received back from the macular tissue at the back of the eye. Wavelengths in the range of 850-1350 nm may have sufficiently low water absorption to allow light to penetrate several cm in a minimally scattering medium such as the eye. The emitted light 39 impinges on the macula 40 and the retinal area 38. The presence of drusen proximate to either the macula 40 or the retina 38 causes the light to be absorbed or attenuated before it returns to the detector 24, indicated by arrow 41.

The sensor 14 may be arranged to emit light with a specific path length into the eye 28. Because the sensor 14 is in a reflectance configuration, the light originating from the emitter 22 first travels into the tissue and is refracted before impinging on the detector 24. For reflectance sensors, the light that passes through the tissue and is related to the drusen levels does not travel directly from the emitter 22 to the detector 24 by the shortest geometric path, but instead travels in a substantially V-shaped configuration through the tissue, as indicated schematically in FIG. 4. The optical distance for such a configuration is the geometric length of the V-shaped path the light follows from the emitter 22 to the detector 24. The path length may be related to the distance d₁ between the emitter 22 and the detector 24. The farther the distance between them, the longer the path length of the emitted light 39. In certain embodiments, d₁ may be in the range of 1 cm-5 cm. Generally, the path length should be sufficient to allow the emitted light 39 to reach structures towards the back of the eye 28, such as the macula 40 and the retina 38, which may be at a distance d₂ from the sensor 14. Depending on how far the sensor 14 is positioned from the eye 28, d₂ may vary. In certain embodiments, d₂ may be in the range of 1 cm-5 cm, for example.

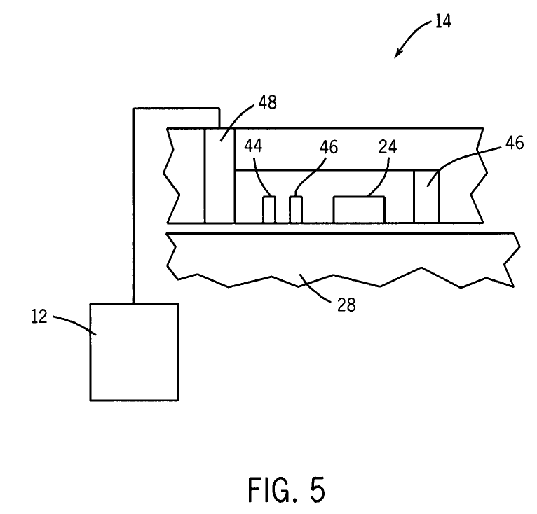

FIG. 5 is a block diagram that is representative of a specific embodiment of the sensor 14 that operates in accordance with present embodiments. Specifically, as illustrated in FIG. 5, the sensor 14 may include a spectrophotometry sensor or photo-sensor that includes a first LED 44, a second LED 46, and a photo-detector 24. It should be noted that while the sensor 14 as depicted merely includes two LEDs, in other embodiments the sensor 14 may include three or more LEDs or other wave emitting devices (e.g., superluminescent diodes (SLD), diode lasers, vertical cavity lasers (VCSELs), resonant cavity LEDs, tunable/scanning lasers, filament bulbs). The sensor 14 may also include a memory 47 to store algorithms and an interface 48 to facilitate communication with the monitor 12. The LEDs 44 and 46 receive drive signals from the monitor 12. The drive signals activate the LEDs 44 and 46 and cause them to emit signals. More specifically, each LED 44 and 46 may be energized individually in an alternating pattern. After the emitted light has been transmitted to the eye 28, the photo-detector 24 receives the dispersed light from the eye 28. The photo-detector 24 then converts the received light into a photocurrent signal, which is eventually provided to a signal processing unit in the monitor 12.

The monitor 12 may utilize data from the photocurrent signal to perform calculations relating to calculation of drusen levels in the eye 28. For example, the monitor 12 may compare measured values with a table of established correlations of drusen levels to determine a lipid or drusen content value for posting as the current retinal tissue lipid or drusen level. Based on the value of the received signals corresponding to the light received by detector 24, a microprocessor will calculate the drusen or lipid concentration using various algorithms. These algorithms utilize coefficients, which may be empirically determined, corresponding to, for example, the wavelengths of light used. In a two-wavelength system, the particular set of coefficients chosen for any pair of wavelengths is determined by one or more values encoded by the memory 47 corresponding to a particular light source in a particular sensor 14. For example, the first wavelength may be a lipid signal wavelength, and the second wavelength may be a water correction wavelength.

In one embodiment, the coefficients may be encoded by one or more passive components, such as a resistor, rather than by an electronic memory 47. For example, multiple resistor values may be assigned to select different sets of coefficients. In another embodiment, the same resistors are used to select from among the coefficients appropriate for an infrared source paired with either a near red source or far red source. The selection between whether the wavelength sets can be selected with a control input. Control inputs may be, for instance, a switch on the monitor, a keyboard, or a port providing instructions from a remote host computer. Furthermore, any number of methods or algorithms may be used to determine lipid or drusen levels, or any other desired physiological parameter. Embodiments of the present techniques may also include algorithms that are derived empirically, based on data from human patients or animal models.

In embodiments in which the sensor emits and detects discrete wavelengths of light rather than a broader range of wavelengths, the algorithm to determine the concentration of drusen may employ a linear or ratiometric combination of measured absorptions at the respective wavelengths. Such combinations are disclosed U.S. Pat. No. 6,591,122, the disclosure of which is hereby incorporated by reference in its entirety. Such algorithms may calculate the quantify of lipid or drusen in the optical path of the light traversing the ocular tissue. The quantity of lipid or drusen may be determined using algorithms where received radiation intensities measured at two or more wavelengths are combined linearly or to form either a single ratio, a sum of ratios or ratio of ratios of the form log [R(λ₁)/R(λ₂)] in which the linear or ratiometric combination depends primarily on the sum of the absorbances of non-heme proteins and lipids in the ocular tissue. To ensure that the linear or ratiometric combination yields estimates of lipid or drusen that are insensitive to variations in the optical path through the eye, where water is the dominant absorber, the lengths of the optical paths through the ocular tissue at the wavelengths at which the reflectances are measured are matched as closely as possible. This matching is achieved by judicious selection of wavelength sets that have similar water absorption characteristics.

The contribution of water to the total absorption may be calculated and corrected by using one or more reference wavelengths. For example, water absorption, such as at wavelengths between 850-1380 nm, may be used as a reference to calculate the total contribution of water absorption to the spectrum. Specifically, water has absorption coefficients of approximately 0.07 cm⁻¹ and 0.53 cm⁻¹ (log₁₀) at the respective fat-absorption peaks of 930 and 1210 nm in this spectral region. Because light is minimally scattered by the structures of the eye, the amount of water traversed by photons emitted from and received by the sensor will primarily vary with the size of the eye, or with the angle at which light is emitted into the eye and detected from the retina. Water absorption in this spectral region contains peaks that are much broader than the fat absorption peaks. The difference between the absorption at a fat absorption peak and at nearby wavelength that is less strongly absorbed by fat, but still has similar absorption by water could be used to compute an indication of the amount of fat (drusen) in the optical path, independent of the amount of water. Alternatively, two reference wavelengths on either side of the fat absorption peak could be used, and the absorptions could be combined from all three wavelengths, to estimate the second derivative of the optical spectrum near the fat absorption peak. Although lipid absorption may be distinguished from water absorption at near infrared wavelengths, in certain embodiments, it may be advantageous to correct for the contribution of water absorption to the total absorption in order to obtain a corrected absorption. After calculating a calibrated drusen level, a processor may instruct a display on the monitor 12 to display a message related to the drusen levels. The message may be a numerical or semi-quantitative indication of the amount of drusen detected in the optical path of the light emitted and received by the sensor. The quantitative indication may, for instance, be a percentage of the mean lipid levels detected macular tissue spectra of “normal”, or healthy subjects, or a percentage of the “upper lipid of normal subjects”, which levels would need to be determined through empirical clinical testing.

Additionally, a message may include an audio and/or visual alarm if the drusen level is greater than or less than an empirically determined threshold. A message may also be a text indicator, such as “DRUSEN LEVELS WITHIN NORMAL RANGE.”

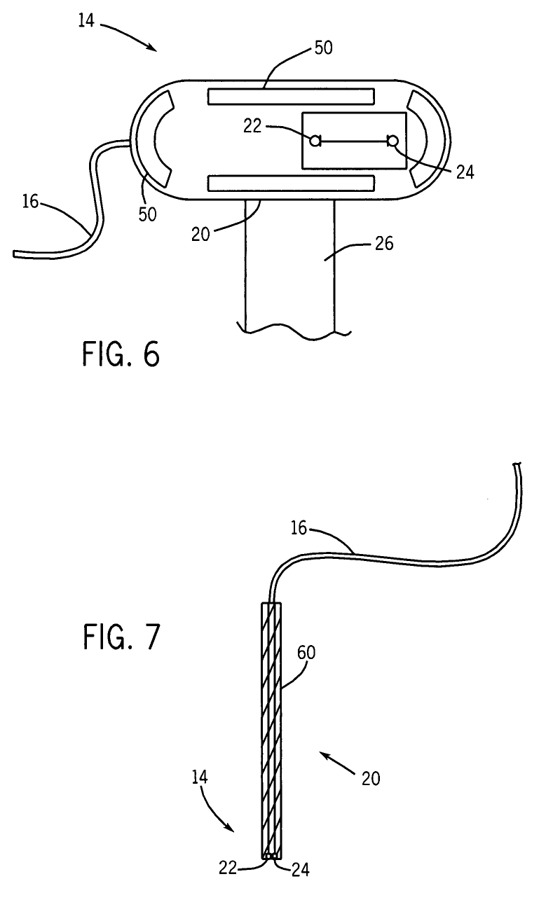

Generally, the lipid or drusen absorbance peaks have widths of about 50 nm, which are fairly close to broad water absorbance peaks. To distinguish between the contributions of water (which makes up most of the tissue that the photons would have to traverse through the eye) and fat (the distinguishing component of drusen), two reference wavelengths may be used, the first a few tens of nm shorter than the fat peak and the second a few tens of nm longer than the fat peak. For example, for fat absorbance peaks of 930 nm and/or 1210 nm, the water reference wavelength may be in the range of 890 nm-910 nm and 950 nm-970 nm, and 1160 nm-1190 nm and 1230 nm-1260 nm respectively. Such a wavelength selection may enable linear or ratiometric combinations of the absorptions at the selected wavelengths that are primarily sensitive to the relatively narrow lipid absorbance peaks and are relatively insensitive to the absorbance of the water in the eye.
As noted, certain aspects of the sensor 14 may also be specifically optimized for a non-invasive application. Generally, such an application may be advantageous for routine eye exams. In a non-invasive embodiment, the body 20 of the sensor 14 may be configured for placement adjacent a patient's eye 28, as illustrated in FIG. 6. Specifically, FIG. 6 shows the attachment-side (i.e., the side configured to couple to the patient) of a non-invasive embodiment of the sensor 14. In this embodiment, the sensor body 20 may include a flexible sheet that conforms to the patient's eye 28. For example, the sensor body 20 may comprise a thin, elongate piece of rubberized material, flexible plastic or woven fibers. Additionally, the sensor body 20 may include cushions or spacers 50 in order to keep the emitter 22 and detector 24 from directly contacting the eye 28. In certain embodiments, these spacers 50 may also be useful for blocking some or all ambient light from reaching the detector 24. Further, the sensor body 20 may be formed from a material that exhibits short-term or long-term biocompatibility to prevent undesired reactions when put in contact with the patient's skin. Additionally, the sensor body 20 may be configured to protect internal components from exposure to elements (e.g., sweat) that might interfere with the function of the internal components.

Further, the sensor 14 may include a positioning stand 26 that may position the emitter 22 and detector 24 at a suitable distance from the eye 28 in order to achieve a predetermined or precalibrated path length based on the distance between the emitter 22 and the detector 24. The positioning information may be stored in an encoder or memory 47, and the stand 26 may be operatively connected to the monitor 12 in order to automate the positioning process. Accordingly, in some embodiments for non-invasive applications, the sensor 14 includes an emitter 22 and detector 24 with a source-detector separation of at least 200 micrometers.

Alternatively, a sensor 14 may include a microneedle structure to allow minimally invasive insertion of a sensor into the eye. FIG. 7 illustrates an exemplary fiber optic sensor 14. The sensor body 20 includes a fiber optic microneedle shaft 60 that may be inserted a short distance into a patient's eye. As illustrated in FIG. 7, one end of the fiber optic microneedle 60 is connected to an emitter 22. The microneedle 60 is also connected to a detector 24 for detecting the light transmitted through the microneedle 60. The light may be transmitted using optical fibers. Such a configuration may provide the advantage of a small, minimally invasive structure that may pierce through the outer layers of a patient's eye to probe the retina 38. The microneedle 60 may thus be sufficiently long to traverse the eye 28 to probe the retina 38 or macula 40. The use of fiber optic sensing elements coupled to the emitter 22 and the detector 24 may be advantageous because they may be configured to have very small optical distances. Thus, the emitter 22 and detector 24 may be in the configuration of a fiber optic bundle with multiple emitting and detecting fibers that are configured to shine light into the tissue. Fiber optic sensing elements may be conventional optical fibers having a light transmitting fiber core that is transparent in the near-infrared range. The fibers may also include a cladding layer (not shown) for preventing or restricting transmission of light radially out of the core, and a protective outer or buffer layer (also not shown). The emitter 22 may also include coupling optics, such as a microscope objective lens, for transmitting light into the fiber.

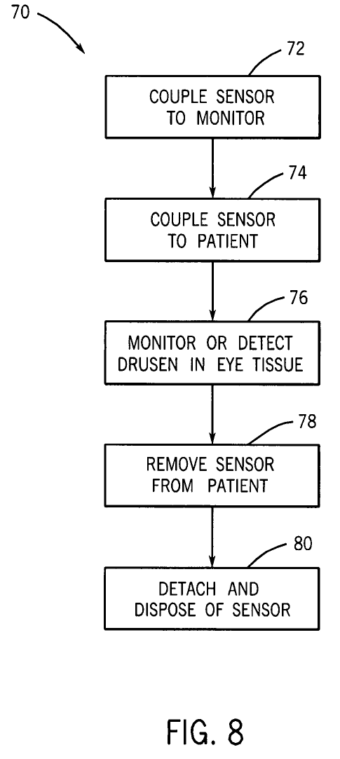

FIG. 8 is a block diagram of a method in accordance with an exemplary embodiment of the present invention. The method is generally designated by reference numeral 70. Block 72 represents attaching or coupling the sensor 14 to the monitor 12. Block 74 represents coupling the sensor to a patient. In certain embodiments, block 74 may include positioning the sensor in front of the patient's eye, as shown in FIG. 2. Alternatively, the sensor 14 may be inserted into the eye, for example with a microneedle. Block 76 represents monitoring or detecting the drusen in and around the macular structure. The monitoring in block 76 may continue for any suitable amount of time depending on the condition of the patient. Block 78 represents removal of the sensor 14 from the patient. Block 80 represents detachment of the sensor 14 from the patient, and disposal of the sensor 14. In an alternative embodiment, all or part of the sensor 14 may be cleaned and reused.

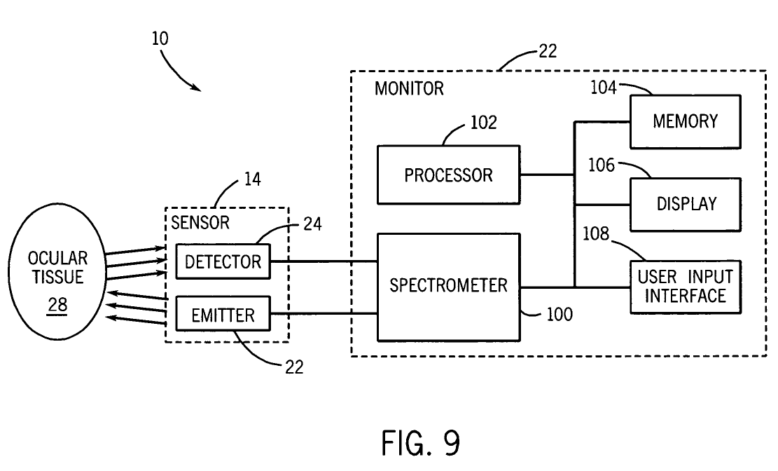

Embodiments of the present techniques may utilize multiple linear regression to calculate the contributions of lipid, water, and/or protein to the absorption spectra. In such embodiment, the system 10 (see FIG. 9) may include a spectrometer 100 configured to emit a range of wavelengths of light into a patient's tissue. The system may also include a processor 102, a memory 104, the display 106, and an input interface 108. More specifically, the system 10 may include components found in oximeters and tissue hydration monitors under development by Nellcor Puritan Bennett LLC of Pleasanton, Calif.

The sensor 14 includes the emitter 22 and the detector 24. Light emission and detection through the sensor 14 may be controlled by the spectrometer 100. Because the emitter 22 is configured to emit a range of wavelengths of light, the emitter 22 may include a plurality of illumination fibers for emitting light into the ocular tissue. The detector 24 may also consist of a plurality of detection fibers and may be configured to transmit light to the spectrometer 100 via the fibers. The detected light from the detector 24 may be transmitted to the spectrometer 100 in the system 10. The spectrometer 100 separates the detected light according to wavelength and converts the intensity to a measure of absorbance to determine an absorbance spectrum. The processor 102 may then perform a multi-linear regression on the measured absorbance spectrum, as described below, using estimated or standardized absorbance spectra of the individual tissue constituents. An algorithm for performing the multi-linear regression, as described below, along with the absorbance spectra information for each of the individual tissue constituents, may be stored in the memory 104. Additional information for use in the multi-linear regression algorithm, such as, for example, the subject's body temperature, may be entered into the system 10 via the input interface 106.

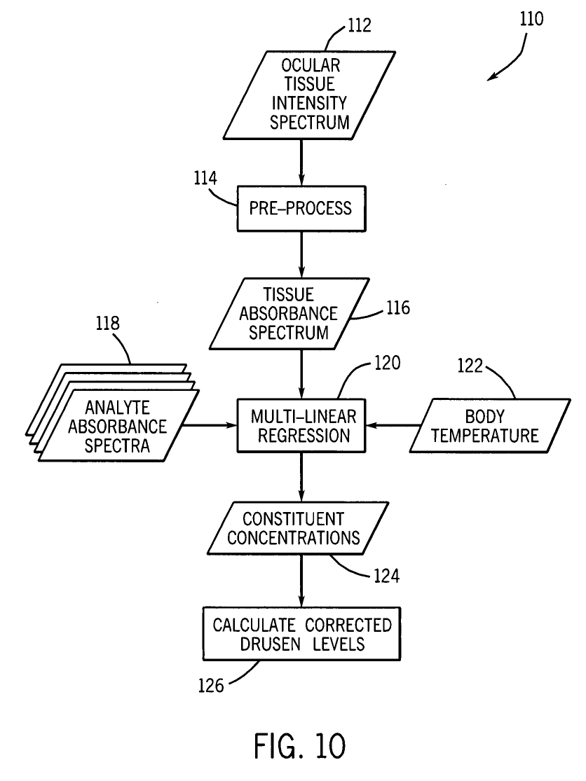

The system 10 may be configured to correct for the water content of the absorption spectrum by performing a multi-linear regression in relation to absorbance spectra of known tissue constituents. FIG. 10 is a flow chart illustrating a process 110 by which water absorption may be corrected. The intensity of light detected by detector 24 may be represented as a tissue intensity spectrum 112. The tissue intensity spectrum 112 may be pre-processed (Block 114), as described below, to produce a tissue absorbance spectrum 116. This tissue absorbance spectrum 116 may be compared to a plurality of analyte absorbance spectra 118 in a multi-linear regression (Block 120). In addition, other factors may be considered in the multi-linear regression (Block 120). For example, a patient's body temperature 122 may be input into the multi-linear regression (Block 120) as described below. The result of the multi-linear regression (Block 120) is the constituent concentrations 124. These constituent concentrations 124 may then be used to subtract out the water absorption.

The conversion of the intensity spectrum 112 to the absorbance spectrum 116 is based on Beer's law:

$${I}_{detected} = {I}_{emitted} {10}^{- l {\sum }_{i} {β}_{i} {c}_{i}} , \qquad ( 1 )$$

where I is the intensity of light, l is the optical pathlength, and bᵢ are cᵢ are respectively the optical extinction coefficient and the concentration of the ith analyte. In accordance with present embodiments, I_{emitted} may be adjusted to account for various factors, such as instrument or sensor factors that affect the accuracy of Equation (1).

In order to perform the multi-linear regression (Block 120) of the ocular tissue absorbance spectrum 116, the absorbance spectra 118 of the main constituents found in the eye may be measured or approximated over the entire near-infrared region (i.e., approximately 1000-2500 nm) or a subset thereof (i.e., 1000-1350 nm). The spectra 118 include a water absorbance spectrum, a protein absorbance spectrum, an oxygenated hemoglobin (HbO₂) absorbance spectrum, and an analyte (i.e. a drusen) absorbance spectrum. Other analytes for which known absorbance spectra may be collected and that may be used in embodiments of the present invention include deoxygenated hemoglobin (Hb); water at different temperatures; known mixtures of water, protein, and lipid; different varieties of proteins (e.g., elastin, albumin, keratin, and collagens); different varieties of lipids (e.g., oleic acid, cholesterol, palmitic acid, corn oil and canola oil); saturated and unsaturated fats; proteins dissolved in deuterium oxide (“heavy water”); and any other analyte representative of known skin constituents. The absorbance spectra 118 may be acquired by measuring light transmitted through a cuvette containing the representative, and desirably non-scattering, analyte.

Referring again to FIG. 10, based on the measured analyte absorbance spectra 118, the concentration of skin constituents may be determined from the tissue absorbance spectrum 116 in the multi-linear regression (Block 120). Multi-linear regression may be employed to determine a linear combination of the known analyte absorbance spectra 118, that best matches the measured ocular tissue absorbance spectrum 116. In other words, the multi-linear regression determines to what extent each tissue constituent contributes to the values of the measured tissue absorbance spectrum 116. The multi-linear regression (Block 120) may be characterized by the following set of equations:

$$\begin{matrix}\begin{matrix}{A}_{{λ}_{1}}^{M} = {C}_{W} {A}_{{λ}_{1}}^{W} + {C}_{P} {A}_{{λ}_{1}}^{P} + {C}_{L} {A}_{{λ}_{1}}^{L} + {C}_{H} {A}_{{λ}_{1}}^{H} + b \\ {A}_{{λ}_{2}}^{M} = {C}_{W} {A}_{{λ}_{2}}^{W} + {C}_{P} {A}_{{λ}_{2}}^{P} + {C}_{L} {A}_{{λ}_{2}}^{L} + {C}_{H} {A}_{{λ}_{2}}^{H} + b\end{matrix} \\ ⋮ \\ {A}_{{λ}_{n}}^{M} = {C}_{W} {A}_{{λ}_{n}}^{W} + {C}_{P} {A}_{{λ}_{n}}^{P} + {C}_{L} {A}_{{λ}_{n}}^{L} + {C}_{H} {A}_{{λ}_{n}}^{H} + b\end{matrix} , \qquad ( 3 )$$

where A is the absorbance, λₙ is the wavelength, C is the concentration of the constituent, b is a wavelength-independent offset, M denotes the measured tissue, W denotes water, P denotes proteins, L denotes lipids, and H denotes oxygenated hemoglobin. Additional terms may be added for other analytes. It should be understood by one skilled in the art that the number of independent equations required to find the unknown parameters (i.e., the constituent concentrations 64 (C) and the offset (b)) is equal to the number of unknown parameters. This system may also be expressed using the following equation:

$${A}^{M} = {CA}^{S} , \\ where \\ {A}^{M} = ( \begin{matrix}{A}_{{λ}_{1}}^{T} \\ {A}_{{λ}_{2}}^{T} \\ ⋮ \\ {A}_{{λ}_{N}}^{T}\end{matrix} ) , \\ C = ( \begin{matrix}{C}_{W} \\ {C}_{P} \\ {C}_{L} \\ {C}_{H} \\ b\end{matrix} ) , \\ and \\ {A}^{S} = ( \begin{matrix}{A}_{{λ}_{1}}^{W} & {A}_{{λ}_{1}}^{P} & {A}_{{λ}_{1}}^{L} & {A}_{{λ}_{1}}^{H} & 1 \\ {A}_{{λ}_{2}}^{W} & {A}_{{λ}_{2}}^{P} & {A}_{{λ}_{2}}^{L} & {A}_{{λ}_{2}}^{H} & 1 \\ ⋮ & ⋮ & ⋮ & ⋮ & ⋮ \\ {A}_{{λ}_{N}}^{W} & {A}_{{λ}_{N}}^{P} & {A}_{{λ}_{N}}^{L} & {A}_{{λ}_{N}}^{H} & 1\end{matrix} ) . \qquad ( 4 )$$

Given the measured tissue absorbance spectrum 116 (A^{M}) and the known analyte absorbance spectra 118 (A^{S}), the concentration 124 (C) of each constituent may be calculated. Because the measured tissue absorbance spectrum 116 and the known analyte absorbance spectra 118 may be represented as matrices, as illustrated in Equation (4), solving for the constituent concentrations 124 may be performed using a suitable matrix manipulation environment, such as, for example, MATLAB®, available commercially from The MathWorks, Natick, Mass. The matrix manipulation environment may, for example, be utilized to find the constituent concentrations 114 (C) in Equation (4) by multiplying each side of the equation by the inverse of the matrix representing the analyte absorbance spectra 118 (A^{S}). The matrix manipulation environment may, for example, be stored in the memory 104 of the system 10 for use by the processor 102.

Equations (3) and (4) illustrate a simple multi-linear regression model which considers only four tissue constituents and a wavelength-independent offset which accounts for variations in light input. Additional factors may be added to the equations to account for observed differences in estimated and actual body fluid metrics. For example, the multi-linear regression model may include a temperature component to account for temperature-dependent changes in hydrogen bonding which affect the width and center frequencies of the water absorbance bands. That is, the patient's body temperature may be measured and used as an input to the model. The effect of temperature on the water absorbance spectrum is due to hydrogen bonds between molecules which decrease as temperature increases. The temperature component of the multi-linear regression model may include adjustment of the known water absorbance spectrum for the measured body temperature and/or use of a specific known water absorbance spectrum corresponding to the measured temperature. Thus, equation (3) may be rewritten as follows:

A_{λ}_{ₙ}^{M}=C_{W}A_{λ}_{ₙ}^{W}(T)+C_{P}A_{λ}_{ₙ}^{L}+C_{L}A_{λ}_{ₙ}^{L}+C_{H}A_{λ}_{ₙ}^{H}+b, (5)

where T is the patient's body temperature, and the known absorbance spectrum of water (A^{W}) is dependent on temperature.

Further adjustments to the multi-linear regression model may consist of, for example, adding a slope factor in addition to the known analyte absorbance spectra (A^{S}) and the wavelength-independent offset, or a factor to account for the reduction in mean photon pathlength that occur with increasing absorption coefficients in those portions of the optical path where scattering occurs, as described in U.S. Patent Application “METHOD AND APPARATUS FOR SPECTROSCOPIC TISSUE ANALYTE MEASUREMENT,” filed on Mar. 5, 2007, by Clark R. Baker Jr., et al., the disclosure of which is incorporated by reference in its entirety.

While the invention may be susceptible to various modifications and alternative forms, specific embodiments have been shown by way of example in the drawings and will be described in detail herein. However, it should be understood that the invention is not intended to be limited to the particular forms disclosed. Rather, the invention is to cover all modifications, equivalents and alternatives falling within the spirit and scope of the invention as defined by the following appended claims.

What is claimed is:

1. A sensor for detecting or monitoring macular degeneration in a patient, comprising:
a sensor body;
an emitter disposed on the sensor body adapted to emit at least one wavelength of light through a patient's retina at a lipid-absorbing wavelength in the range of 915 nm to 940 nm or in the range of 1160 nm to 1230 nm; and
a detector disposed on the sensor body adapted to detect the at least one wavelength of light.

2. The sensor, as set forth in claim 1, wherein the emitter comprises at least one light emitting diode.

3. The sensor, as set forth in claim 1, wherein the detector comprises at least one photodetector.

4. The sensor, as set forth in claim 1, wherein the at least one wavelength of light is approximately 930 nanometers.

5. The sensor, as set forth in claim 1, wherein the at least one wavelength of light is approximately 1210 nanometers.

6. The sensor, as set forth in claim 1, comprising a calibration element adapted to provide at least one signal related to at least one physical characteristic of the sensor.

7. The sensor, as set forth in claim 6, wherein the calibration element comprises a coded resistor or an electrically erasable programmable read-only memory.

8. The sensor, as set forth in claim 1, wherein the sensor comprises an optical fiber.

9. The sensor, as set forth in claim 1, wherein the sensor body is adapted to conform to a patient's eye.

10. The sensor, as set forth in claim 1, wherein the sensor comprises a microneedle.

11. The sensor, as set forth in claim 1, wherein the emitter is adapted to emit a second wavelength related to a reference signal, and wherein the detector is adapted to detect the second wavelength.

12. The sensor, as set forth in claim 11, wherein the second wavelength related to a reference signal is between 850 nm and 1350 nm.

13. The sensor, as set forth in claim 11, wherein the emitter is adapted to emit a third wavelength, and wherein the detector is adapted to detect the third wavelength.

14. The sensor, as set forth in claim 13, wherein the second wavelength related to the reference signal is shorter than the first wavelength, and wherein the third wavelength related to the reference signal is longer than the first wavelength.

15. The sensor, as set forth in claim 1, wherein the emitter is adapted to emit a range of wavelengths between 915 nm to 940 nm or between 1160 nm to 1230 nm.

16. A method of detecting or monitoring macular degeneration in a patient, comprising:
emitting at least one wavelength of light into a patient's eye at a wavelength in the range of 915 nm to 940 nm or in the range of 1160 nm to 1230 nm;
detecting the light after dispersion by drusen in the eye; and
determining an amount of drusen based on the detected light.

17. The method, as set forth in claim 16, emitting the least one wavelength of light comprises inserting a microneedle into the patient's eye.

18. The method, as set forth in claim 16, comprising emitting a second wavelength related to a reference signal, and wherein the detector is adapted to detect the second wavelength.

19. The sensor, as set forth in claim 18, wherein the second wavelength related to a reference signal is between 850-1380 nm.

20. The method, as set forth in claim 18, comprising emitting third wavelength, and wherein the detector is adapted to detect the third wavelength.

21. The method, as set forth in claim 20, wherein the second wavelength related to the reference signal is shorter than the first wavelength, and wherein the third wavelength related to the reference signal is longer than the first wavelength.

22. The method, as set forth in claim 16, comprising emitting a range of wavelengths between 915 nm to 940 nm or between 1160 nm to 1230 nm.

23. A system for detecting or monitoring macular degeneration in a patient, comprising:
a sensor comprising:
a sensor body;
an emitter disposed on the sensor body adapted to emit at least one wavelength of light through a patient's retina at a wavelength in the range of 915 nm to 940 nm or in the range of 1160 nm to 1230 nm; and
a detector disposed on the sensor body adapted to detect the at least one wavelength of light; and
a monitor operatively connected to the sensor.

24. The system, as set forth in claim 23, wherein the emitter comprises at least one light emitting diode.

25. The system, as set forth in claim 23, wherein the detector comprises at least one photodetector.

26. The system, as set forth in claim 23, wherein the at least one wavelength of light is approximately 930 nanometers.

27. The system, as set forth in claim 23, wherein the at least one wavelength of light is approximately 1210 nanometers.

28. The system, as set forth in claim 23, comprising a calibration element adapted to provide at least one signal related to at least one physical characteristic of the sensor.

29. The system, as set forth in claim 28, wherein the calibration element comprises a coded resistor or an electrically erasable programmable read-only memory.

30. The system, as set forth in claim 23, wherein the sensor comprises an optical fiber.

31. The system, as set forth in claim 23, wherein the sensor body is adapted to conform to a patient's eye.

32. The system, as set forth in claim 23, wherein the sensor comprises a microneedle.

33. The system, as set forth in claim 23, wherein the emitter is adapted to emit a second wavelength related to a reference signal, and wherein the detector is adapted to detect the second wavelength.

34. The system, as set forth in claim 33, wherein the second wavelength related to a reference signal is between 850-1380 nm.

35. The system, as set forth in claim 33, wherein the emitter is adapted to emit a third wavelength, and wherein the detector is adapted to detect the third wavelength.

36. The system, as set forth in claim 35, wherein the second wavelength related to the reference signal is shorter than the first wavelength, and wherein the third wavelength related to the reference signal is longer than the first wavelength.

37. The system, as set forth in claim 23, wherein the emitter is adapted to emit a range of wavelengths between 915 nm to 940 nm or between 1160 nm to 1230 nm.
